(19) United States
(12) Patent Application Publication
(10) Pub. No.: US 2026/0069141 A1
(43) Pub. Date: Mar. 12, 2026

(54) A SYSTEM FOR PROJECTING A SELECTIVE OPTICAL PATTERN OF INTEREST INTO A HUMAN EYE

(71) Applicant: La Science SAS, Paris (FR)

(72) Inventors: Guillaume Buc, Bures sur Yvette (FR); Jacques Feldmar, Saint Mande (FR); Giulia Lia Beatrice Spampinato, Fontainebleau (FR)

(73) Assignee: La Science SAS, Paris (FR)

(21) Appl. No.: 19/101,736

(22) PCT Filed: Aug. 10, 2023
(86) PCT No.: PCT/EP2023/072219

§ 371 Date: Feb. 6, 2025

Related U.S. Application Data
(63) Continuation of application No. PCT/EP2022/072612, filed on Aug. 11, 2022.

(30) Foreign Application Priority Data
Aug. 11, 2022 (WO) PCT/EP2022/072612

(51) Int. Cl. A61B 3/14; A61B 3/12

(52) U.S. Cl. CPC A61B 3/14; A61B 3/12

(57) ABSTRACT

The invention relates to a system for projecting an optical pattern of interest into a human eye in a selective manner. In particular, the invention relates to a system for supporting the identification of an optical pattern of interest and to project said pattern of interest onto a predefined retinal area of the human eye. Accordingly, a system (10) for projecting an optical pattern of interest (28) onto a predefined retinal area (22) of a human eye is suggested, comprising a camera (12) for capturing an image, a projector device (14) for projecting a light beam (20) into a human eye based on an inputted signal, and a processor (16) being in communication with the camera (12) and the projector device (14), the processor (16) being adapted to convert a portion of the captured image into a corresponding signal and to output said signal to the projector device (14). According to the invention, the processor (16) is further adapted to detect a presence of a pointing element (34) in the captured image and to convert the portion of the captured image corresponding to a predefined region (30) of the image relative to the detected pointing element (34) and comprising at least in part the pattern of interest (28).

### TECHNICAL FIELD

The invention relates to a system for projecting an optical pattern of interest into a human eye in a selective manner. In particular, the invention relates to a system for supporting the identification of an optical pattern of interest and to project said pattern of interest onto a predefined retinal area of the human eye.

### TECHNOLOGICAL BACKGROUND

Retinal malfunction, particularly caused by degenerative retinal diseases, is a leading reason for visual impairment or even blindness.

For at least partially restoring a patient's visual function, it is known to modify or, rather, to adapt a retinal area by making use of a retinal implant or in other words a retinal prosthesis. In this regard, several different types of retinal implants are known, which are based on different working principles.

Retinal implants have in common that they are usually placed suprachoroidally, epiretinally or subretinally in the eye of the patient, such that they can replace i.e. effect the damaged photoreceptors. In this regard, information about a visual scene is captured with a camera and then transmitted to an electrode array implanted in the retina.

Among common retinal implants, implants are known which comprise skin-penetrating wires. These wires introduce risks of infection and scarring. Thus, more modern implants use different wireless techniques, for instance by delivering power and visual information through inductive coils. Furthermore, it is known to deliver power inductively and visual information optically through the pupil of the eye, or to deliver both visual information and power optically.

A particularly beneficial type of wireless information transfer retinal implants is based on projecting stimulation patterns of infrared light into the eye. When the gaze direction is such that some part of the implant is illuminated by part of the stimulation pattern, the implant converts that part of the signal to electrical current that stimulates the retina accordingly. The retinal implant is typically an array composed of stimulation electrodes or pixels. Each pixel has one or several photodiodes that capture the light delivered from a visual processor and converts it into electrical current for stimulation.

Several implant arrays can be placed in the subretinal space, typically in or close to the foveal area.

An alternative approach is the use of optogenetic applications to modify or improve a targeted retinal area in the eye, such as reactivating the photosensibility of the targeted area of the retina. Optogenetics refers to the combination of genetics and optics to control well-defined events within specific cells of living tissue. Optogenetics consists in (i) genetically modifying target cells in order to render them sensitive to light by the expression of exogenous photoreactive proteins in cellular membrane and (ii) providing an illuminating device able to provide light to said photoreactive proteins.

The projecting of light or a light beam may be provided within a particular wavelength range having a specific irradiance adapted for the activation of the exogenous photoreactive proteins in the cellular membrane of the modified retinal area, or of the retinal implants. Accordingly, the light beam may be output e.g. in the infrared range, thereby ensuring that areas of the retina that have not been modified or have not been adapted with an implant are not affected by the projected light.

Furthermore, for projecting light or a light beam into a human eye, it is known to use a projector device that projects the light beam onto the human eye, wherein the light beam may be adapted to the size of the pupil of the human eye. This ensures that essentially the entire light beam may be provided to the retina of the human eye and the information received from a camera input and converted into the light beam may be provided to the largest extent.

### SUMMARY OF THE INVENTION

Starting from the known prior art there is a need to further facilitate that an optical pattern of interest may be perceived by a patient in a convenient manner.

According to the invention it has been recognized that even with the provision of a proper light beam size to enter the pupil of the human eye it may be difficult for a patient to obtain the desired visual information. This results from the fact that a field of view of a camera is typically larger than a field of view of the corresponding light beam, such that the light beam cannot provide the entirety of information available within a captured image. To facilitate that a patient may focus on a pattern of interest, the light beam and the camera are typically aligned in a predefined manner, such that a movement of the camera results in a corresponding change of information perceived by the patient.

However, the size of a retinal implant or modified retinal area of a human eye is typically smaller than the size of the light beam, such that the optical pattern of interest may not or only be partially perceived by the patient. This may lead to uncertainty at the patient end, in particular, since the resolution of a retinal implant or capability of a modified retinal area to transmit signals with appropriate resolution is generally low. If the portion of the light beam projecting on predefined retinal area (e.g. retinal implant or modified retinal area) hence does not allow identification of the pattern of interest, the patient may be confused or even be dazzled, e.g. in case of high contrasts or intensities captured in the image and provided by the projected light beam.

It is hence an object of the present invention to provide an improved system for projecting a pattern of interest onto a retinal area of a human eye, in particular to facilitate a focusing and/or centering of the system and predefined retinal area with a pattern of interest.

The above object is solved by means of a system for projecting an optical pattern of interest onto a predefined retinal area of a human eye comprising the features of claim 1. Further preferred embodiments are presented in the dependent claims, the description and the Figures.

Accordingly, in one aspect, a system for projecting an optical pattern of interest onto a predefined retinal area of a human eye is suggested, comprising a camera for capturing an image, a projector device for projecting a light beam, preferably a pulsed light beam, into a human eye based on an inputted signal, and a processor being in communication with the camera and the projector device, wherein the processor is adapted to convert a portion of the captured image into a corresponding signal and to output said signal to the projector device. According to the invention, the processor is further adapted to detect a presence of a pointing element in the captured image and to convert the portion of the captured image corresponding to a predefined region of the image relative to the detected pointing element and comprising at least in part the pattern of interest.

The present inventors have identified that the patient, even in the case of a partial loss of central vision and/or peripheral vision, may use remaining vision to align the camera with a pattern of interest. For example, the patient may still use the peripheral vision in case of a full or partial loss of central vision, and, vice versa, may use at least parts of remaining central vision, if sufficient for aligning the camera. However, misalignments of the camera, the light beam, and/or the predefined retinal area may result in the perception of other visual information that is not of interest or may even potentially confuse the patient. By providing the pointing element, the patient is assisted in finding the pattern of interest, when the pointing element is not at the desired position within the field of view of the camera.

This has been found to be further facilitated by the hand eye coordination of the patient, enabling the patient to either move the pointing element or adapt the orientation of the head and/or eye in case a centering along a common optical axis of the camera, light beam, and/or predefined retinal area is required or desirable for improved resolution and optical quality of the perceived pattern of interest. Even in the absence of such remaining vision, i.e. in case of full vision loss or blindness, such intuitive hand coordination and movement of the pointing element may be used to facilitate finding the pattern of interest.

The “predefined retinal area” preferably corresponds to a position and size of a retinal implant or a modified retinal area (e.g. a reactivated retinal area or modified to restore photosensitive behavior through modification by optogenetics, such as for damaged photoreceptors which have been replaced or reactivated). However, “the predefined retinal area” may also constitute a native portion of the retina of the patient remains functionally intact, yet may not always be aligned with the pattern of interest. Under such circumstances, the “predefined retinal area” may correspond to said portion of the retina, such that the part of the pattern of interest being projected is perceived by said portion of the retina, e.g. with improved optical quality.

The conversion of a portion of the captured image corresponding to a predefined region of the image relative to the detected pointing element furthermore provides that the hand eye coordination of the patient and any potentially remaining vision of the patient may be optimally combined, such that the pattern of interest may be more easily identified and perceived by the patient.

In this regard, the predefined region is preferably adjacent to a predefined end surface of the pointing element. In particular, the predefined region may be directly above the predefined end surface in the image, such that a lateral movement of the camera and corresponding projection of the light beam do not interfere with the presence of the pointing element. In other words, this may provide a more fluent identification of the pattern of interest or a changing pattern of interest, e.g., when reading a document and following the natural movement of a patient's head. Alternatively, the position of the predefined region may be arranged laterally with regard to the (predefined) end surface in the image, wherein a movement of the pointing element in a lateral direction defines the subsequent predefined region in the lateral direction.

In other words, the predefined end surface may point towards the predefined region, e.g. in a longitudinal direction defined by a longitudinal axis of the pointing element, and a movement of the pointing element and corresponding predefined end surface may be understood by the skilled person and perceived by a patient as a dragging or pushing movement of the predefined region and pattern of interest comprised therein.

The processor may be adapted to identify the predefined end surface (of the pointing element) by means of a size, a shape, a texture, a material or tissue type, a level of kurtosis, one or more principal axes, and/or a color of the end surface. For example, the pointing element may be provided as a hand-held pointing device that is optionally included in the system, wherein the pointing device may have e.g. a pen shape or is a pen, marker, or pencil. In such case, the pointing device may comprise straight edges or contours and comprise a tip having a particular curvature and/or color, wherein the identification of the tip may optionally be facilitated by the identification of a main longitudinal axis and/or a texture and/or color of the tip in the image.

In order to identify the predefined end surface, the processor may use one or more algorithms that use image processing and detects one or more of the above characteristics, e.g. using one or more feature recognition algorithms. The predefined end surface (of the pointing element) may then be identified using e.g. machine learning and/or neural networks and/or may use a look-up table or database to match one or more of the identified characteristics with known values or ranges for said characteristics.

The pointing device may furthermore be a dedicated object, preferably in the form of a digital pencil. Advantageously, the processor is then adapted to be communicatively coupled to the pointing device and to determine the predefined region and/or the pattern of interest in the image based on a received orientation information of the pointing device. The pointing device, e.g. the digital pencil, may be equipped with one or more sensors, e.g. (pressure) sensors indicative of the orientation and or interaction with an object of interest such as a hand-held document. The hence received orientation enables a more precise identification of the predefined end surface and provides that the processor may be adapted to adjust the positioning of the predefined region based on the received orientation so as to improve the identification of the pattern of interest.

Alternatively, the pointing element may be provided as one or more fingers of the patient, wherein the predefined end surface is preferably a fingertip. Although this does not provide a communicative coupling, this may nevertheless provide the processor with sufficient information to optionally determine an orientation of the finger. For example, a particular finger, e.g. an index finger, may be identified as the pointing element, wherein a relative angle or distance to one or more adjacent fingers or to the adjacent wrist may enable a calculation or an estimate of the orientation of the index finger and fingertip. Such estimate or calculation may e.g. be based on historical data or on a database with corresponding entries and known orientations or angles of fingers and fingertips. Furthermore, the estimate or calculation may be iteratively improved, e.g. based on machine-learning and a corresponding algorithm.

The predefined end surface (of the pointing element) may furthermore be uniquely identifiable. As described above, the end surface may correspond to a tip of a dedicated object such as a pen or digital pencil, but may also correspond to a fingertip, e.g. of the right or left index finger of the patient. The unique identification ensures that no inadvertent visual information is provided to the patient by means of the light beam in case another object, which is not intended for providing the pattern of interest, or another finger, which may be present to stabilize or hold a document, is within the field of view of the camera. In case of e.g. a fingertip, further features such as biometric characteristics may be retrieved from the captured image.

A size of the predefined region may be delimited and/or defined by a size of the light beam, wherein the processor may be adapted to align the predefined region with the end surface of the pointing element.

The alignment may e.g. be provided as a centering of the predefined region with respect to the predefined end surface. Such centering may support a natural viewing experience, e.g. when following the content above a fingertip when reading, and may facilitate minor corrections of the position of the pointing element and corresponding predefined end surface.

The alignment may also be an offset of the center of the predefined region with regard to the predefined end surface. This ensures that the full size of the light beam may still be used with potentially useful visual information in the case where the pointing element is positioned at an edge region of the field of view of the camera. In other words, the predefined region is aligned with the predefined end surface in such a manner to avoid an extension beyond the camera field of view.

These examples of alignment are not limiting and may be provided as required based on the position of the pointing element. That is, if an offset of the pointing element to the edge of the camera field of view is sufficient, the alignment may be provided as a centering of the predefined region with respect to the predefined end surface, whereas a position close to the edge of the camera field of view may automatically cause a shifting of the predefined region relative to the predefined end surface of the pointing element.

The system is to be understood as a portable system and, in particular, may comprise a spectacles frame and/or comprise a headband and/or ear stoppers to hold the camera and the projector device.

The processor may be provided on the support device or frame, e.g. in an integrated manner with the camera and/or projector device. However, the processor may also be provided separately as a portable device, which is communicatively coupled to the camera and the projector device. Thereby, the weight of the components on the support device or frame may be reduced.

The camera is preferably positioned in front of the projector device, wherein a main axis of the camera may be aligned with a main axis of the light beam, preferably concentrically. Preferably, the camera and the projector device are arranged in line facing opposite sides of the support device or frame. Thereby, it can be ensured that when a person equipped with the projector device is looking straight ahead, the person may see exactly what is straight ahead of him. In other words, it may hence be easier for the person to focus on a point of interest and moreover to orient in space.

However, it may not always be the case that the pointing element is in the center of the camera field of view, i.e. is aligned with an optical axis with the camera and/or the projector device. While this may still provide that at least a portion of the pattern of interest is perceivable by the patient, this may reduce the corresponding resolution, if the pointing element and pattern of interest are not in focus. Furthermore, if the pointing element is positioned at a periphery of the patient or, rather, at a periphery of the patient's vision, continuous or fluent progression of the pointing element, e.g. following a line of text, may be difficult due to a less defined hand eye coordination in the periphery.

The system is therefore preferably adapted to indicate that the pattern of interest is not centered and an alignment with the optical axis should be envisaged.

Accordingly, the processor is preferably adapted to determine a first offset of the pointing element to a central optical axis of the camera and/or a central optical axis of the light beam, wherein the processor is further adapted to base the signal on said first offset and to define a second offset of the predefined region or the pattern of interest to the center of the light beam corresponding to said first offset in said signal.

Alternatively, or in addition, the processor may also be adapted to determine a first offset of the predefined retinal area to a central optical axis of the light beam and/or central optical axis of the camera based on a received orientation information of the predefined retinal area, wherein the processor is adapted to base the signal on said first offset and to define a second offset of the predefined region or the pattern of interest to the center of the light beam corresponding to said first offset in said signal.

Hence, a determined offset of the pointing element to a central optical axis of the system may cause that e.g. the pattern of interest is perceived by the patient with a corresponding shift based on the second offset, indicating that the pointing element is not properly centered. Thereby, the patient is encouraged to move its head and the worn or attached system, such that the optical axis is brought into alignment with the position of the pointing element and a proper perception and focus may be provided.

By the same token, the patient may move his eye with the predefined retinal area while attempting to identify a pattern of interest, deliberately or inadvertently, which may cause the predefined retinal area to be at least partially offset with regard to the projected light beam. In such case, a similar shift will be perceived by the patient, encouraging the patient to move the eye and to bring the predefined retinal area in alignment with the light beam. Although the size of the light beam may exceed the size of the predefined retinal area, the patient may be able to differentiate between these scenarios by perceiving reduced visual information and/or a reduced intensity, when the predefined retinal area is positioned, at least in part, outside of the projected light beam.

In order to receive information regarding the orientation of the predefined retinal area, the projector device may comprise a detachable eye observation module for observing at least a part of the retina, preferably a part of the retina at which the light beam is targeted, wherein preferably, the eye observation module and the light beam comprise the coincident optical axes. The eye observation module may be attached to the projector device and may be used for observing the predefined retinal area and, in particular, the retinal implant implanted in the retina.

In addition, the eye observation module may be adapted to observe the projected pattern on the retina and/or may be adapted for adjusting, aligning and/or centering the light beam projected by the projector device into a patient's eye pupil and onto a retina, more particularly the predefined retinal area, and specifically the retinal implant of the patient.

Hence, the processor may also be adapted to determine a position or orientation of the pupil or eyeball of the patient relative to the light beam, e.g. by using an optical sensor integrated in or attached to the projector device. Based on the determined position, the processor may output a control signal to the projector device to accordingly modify the position and/or orientation of the projector device so as to align the light beam with the pupil of the patient. Thereby, it may be ensured that the light beam is efficiently directed towards the retina, i.e. the predefined retinal area. Thereby, the size of the light beam penetrating the pupil is optimized.

To enable such modification and alignment of the projector device, the projector device may be provided with an alignment device. The alignment device is motorized and dynamic and may e.g. be coupled with an eye observation module, preferably an eye position sensor, allowing direction of the projection to follow the eye and/or pupil direction. Preferably, the eye observation module is configured as an eye tracker device which monitors the eye and/or pupil position and angle and provides the alignment requirement to the alignment device. The alignment device is typically configured to automatically adjust the position and angle of the projected light beam, for instance with micromirrors or piezoelectric micro-motors, to ensure a correct alignment to be carried out automatically and regularly. Thus, the alignment device may include a motorized unit configured to couple the direction of projection with an eye and/or pupil direction as monitored by an eye observation module, preferably an eye tracking device, wherein the eye observation module is preferably embedded in the projection device.

Preferably, the processor is further adapted to crop the predefined region or the pattern of interest in accordance with the second offset and the size of the light beam.

For instance, the pattern of interest may be perceived off center and be cropped compared with a centered perception. In other words, when reading a document, one or more characters may appear to be at least partially be cut off while the characters are aligned with one or more edges of the predefined region, e.g. aligned at a corner position.

Thereby, the size of the received visual information remains essentially the same, but the portion of the predefined region or pattern of interest corresponding to visual information is reduced to indicate that a misalignment exists, motivating the patient to align e.g. the camera and corresponding head position of the patient with the pointing device and/or the predefined retinal area with the optical axis of the system or the position of the light beam at the retina.

The processor may also be adapted to provide the signal defining a predefined and/or standardized pattern for a portion of the light beam not corresponding to the predefined region or the pattern of interest. Preferably, in comparison with the pattern of interest, the predefined and/or standardized pattern may be a pattern with reduced brightness, a blurred pattern, a pattern with reduced resolution, an essentially homogeneous pattern, and/or an essentially continuous pattern.

Such predefined and/or standardized pattern ensures that the patient may always perceive the same size of the light beam and corresponding visual information, which may reduce insecurity regarding the functionality of the system as a whole. Furthermore, this may provide that in case of a misalignment of the predefined retinal area, the patient may more easily align the predefined retinal area with the position of the light beam, since the size of the light beam is not reduced. Accordingly, only minor eye movements may be required to bring the position of the eye in the predefined (natural seeing) position.

In order to facilitate the alignment and centering of the camera with the pointing element and/or of the predefined retinal area with the light beam, the predefined and/or standardized pattern may comprise one or more guidelines originating from the predefined region or the pattern of interest and indicating a direction for centering the camera and/or the predefined retinal area. Such guidelines may be provided as having a perceivable yet not disturbing light intensity that is preferably in contrast with the remaining portion of the predefined and/or standardized pattern. The one or more guidelines may each be connected with a different outer position of the pattern of interest or predefined region and form a respective angle representative for the second offset. The one or more guidelines may be continuously adapted based on adjustments of the camera position or of the position of the predefined retinal area to facilitate a correct positioning and alignment.

Instead of lines, other guiding patterns may also be provided. For example, alternatively, or in addition, a perceivable intensity gradient may be provided, wherein the highest light intensity may correspond to or be adjacent to the pattern of interest.

As described above, the visual information available to the camera may be much more than may be perceived by the patient using the light beam and the corresponding predefined retinal area. This may be due to the size of the predefined retinal area on the one hand and the amount of information that may be processed or transmitted in subsequent stimulation of the corresponding nerve end. For example, a retinal implant may provide a limited resolution of between e.g. 400 and 2500 pixels.

Accordingly, even in case of proper alignment of the camera and the predefined retinal area, the patient may have difficulties to identify the context and interpret the pattern of interest. For example, when the patient is attempting to read a document, such as a book or newspaper, objects such as images, bright areas, and/or patterns that are out of focus that surround text may render it more difficult to identify the relevant information.

Hence, to facilitate identification of the relevant information, it may be provided that a portion of the signal corresponding to the predefined region only defines the pattern of interest.

Thereby, essentially only the pattern of interest within the predefined region may be perceived by the patient and other, not relevant information, may be faded out. The pattern of interest may be identified by the processor using one or more feature recognition algorithms and/or particular shapes or brightnesses may be automatically identified as potentially not relevant, i.e. not corresponding to the pattern of interest. To further increase the perceivability of the pattern of interest or portion thereof, the pattern or object of interest may, alternatively or in addition, be converted so as to provide an abstract representation thereof, e.g. by modifying the brightness and/or contrast and/or by outputting the pattern of interest in a standardized manner, e.g. a predefined text or image layout.

The portion of the predefined region adjacent the pattern of interest may also be provided with a standardized and/or predefined pattern, which may be advantageous e.g. to facilitate a reading flow, when accordingly moving the pointing element and the camera.

### BRIEF DESCRIPTION OF THE DRAWINGS

The present disclosure will be more readily appreciated by reference to the following detailed description when being considered in connection with the accompanying drawings in which:

FIG. 1 shows a schematic depiction of a system according to the invention and its interrelationship with its surroundings and a predefined retinal area;

FIG. 2 shows a schematic depiction of different sizes of a field of view of the camera, the light beam and an image at the predefined retinal area;

FIGS. 3A and 3B show a schematic depiction of the processing of a captured image and its content in the absence of a pointing element; and

FIGS. 4A to 4D schematically show the processing of a captured image and its content in the presence of a pointing element and upon movement.

### DETAILED DESCRIPTION OF PREFERRED EMBODIMENTS

In the following, the invention will be explained in more detail with reference to the accompanying figures. In the Figures, like elements are denoted by identical reference numerals and repeated description thereof may be omitted in order to avoid redundancies.

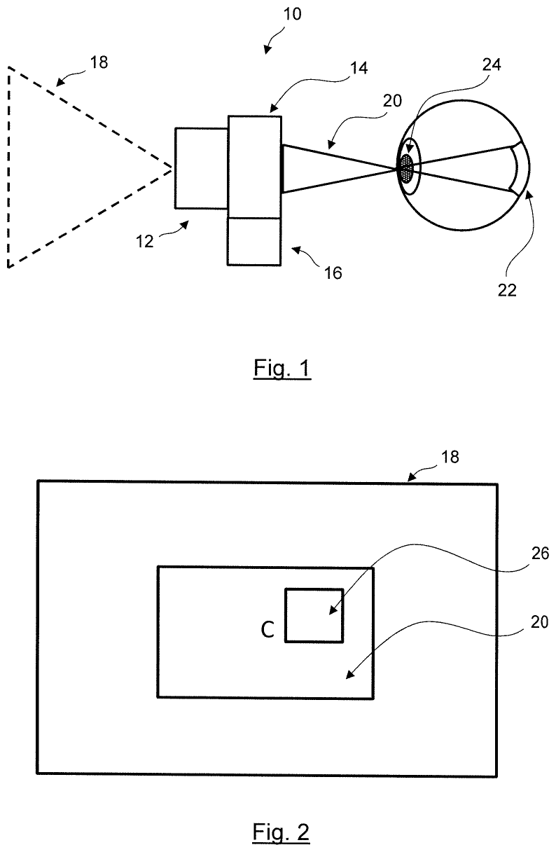

In FIG. 1 a schematic depiction of a system 10 according to the invention is shown. The system 10 comprises a camera 12 and a projector device 14, which are coupled to each other via a processor 16. The processor 16 is shown as being in close arrangement with the other components of the system 10 and may e.g. be attached to or integrated in the projector device 14. Alternatively, however, the processor 16 may also be provided as a separate component so as to distribute the weight of the system 10. This may facilitate the portability of the system 10, since the camera 12 and projector device 14 are preferably attached to a head-worn frame, such as a spectacles frame, wherein experiencing additional weight may be inconvenient for a user or patient. In such separate arrangement, e.g. carried in a pocket or by a body-worn strap, communication between the camera 12, the processor 16, and the projector device 14 may be ensured by means of corresponding wiring or by a wireless functionality, e.g. in a wireless configuration.

In operation, an image is captured by the camera 12, wherein the image is based on the field of view 18 of the camera 12 and a set focus of the camera 12, potentially also a focal distance and/or aperture of the camera. In the image, a pattern of interest such as written text information or other visual content that may be of interest to the user or patient. The image or sequence of images is then provided to the processor 16, which receives the image(s) as an input signal. The processor 16 is adapted to convert a portion of the captured image into a corresponding signal and to output said signal to the projector device 14. In other words, the signal outputted by the processor 16 forms an input signal for the projector device 14, wherein the projector device 14 is adapted to project a light beam 20, preferably a pulsed light beam, into a human eye based on an inputted signal.

The arrangement of the projector device 14 and the size of the light beam 20 are such that the light beam 20 enters the human eye via the pupil 24, preferably in such a manner that the entire light beam 20 may be provided through the pupil 24. After entry and passing through the eye lens, the light beam 20 progresses towards the retina and is projected to a predefined retinal area; in the present non-limiting example a modified or, rather, predefined retinal area 22 comprises a retina implant.

The system 10 thereby enables that visual information obtained by the camera 12 may be processed by the processor 16 and provided to the patient by means of the corresponding light beam 20 projected by the projector device 14. At the retina implant, the received light, preferably pulsed infrared light, is then converted into electrophysiological signals. These electrophysiological signals then stimulate an adjacent nerve end of the patient, such that the patient may perceive the processed visual information, at least in part, which may be considered as a modified or, rather, predefined retinal area image.

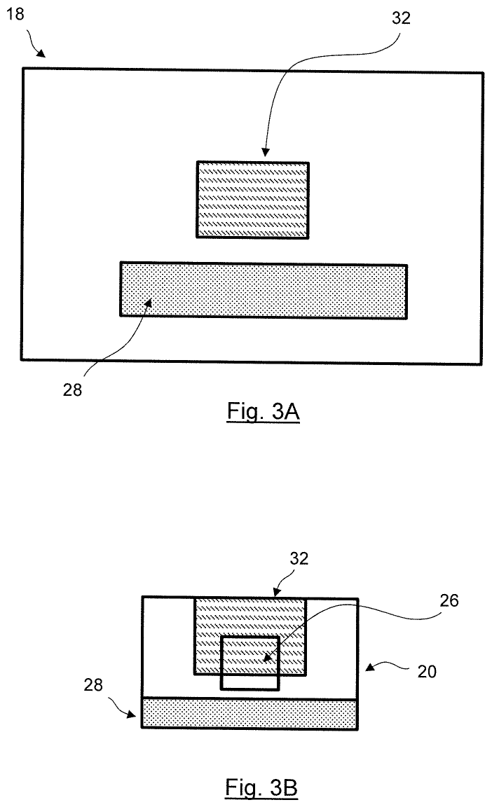

As shown in FIG. 2, a discrepancy exists between the field of view 18 of the camera 12, on the one hand, and the size of the light beam 20, on the other hand, as schematically depicted. In this example, the light beam 20 and the field of view 18 of the camera 12 are aligned along a common optical axis, i.e. in a concentric manner. From the example, it also follows that the predefined retinal area image is smaller than the size of the light beam 20. Furthermore, the modified or, rather, predefined, retinal area image 26 is not perfectly aligned with the optical axis of the camera 12 and the light beam 20. Instead, a slight offset towards the right and the upper surface is present. This may be due to deliberate or involuntary eye movements, resulting in a relative offset of the modified or, rather, predefined retinal area 22 to the center of the light beam 20. As described above, the smaller relative size of the modified or, rather, predefined, retinal area image 26 and any offset to the center position of the eye may render it difficult for the patient to identify a pattern of interest, i.e. distinguish between relevant and not relevant visual information. This situation is aggravated by a typically low resolution of the retinal implant.

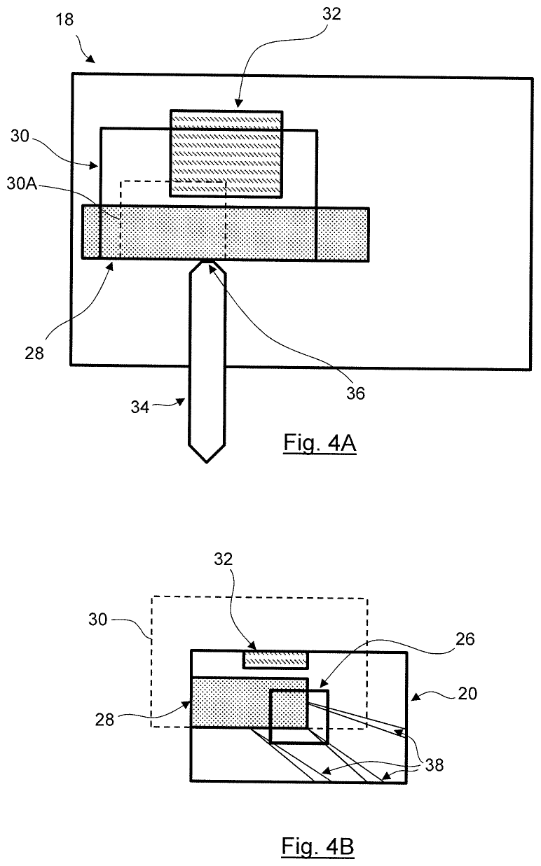

The visual information may furthermore be difficult to identify due to the large amount of available visual information. An example of such situation is schematically shown in FIGS. 3A and 3B. In this example, the camera 12 and light beam 20 are properly aligned and a patient is looking towards a particular area, wherein a pattern of interest 28 is present. As shown, within the field of view 18 of the camera 12 an object 32, not being of particular interest, is also present.

With a properly aligned retinal implant, the modified or, rather, predefined retinal area image 26, i.e. the visual information perceived by the patient corresponds to the center of the image captured by the camera 12. Accordingly, as shown in FIG. 3B, the patient in such case will only perceive part of the object 32, which may e.g. be of high contrast and/or brightness. This in turn could dazzle the patient and result in an orientation loss or loss of motivation to identify the actual pattern of interest 28, situated below the object 32.

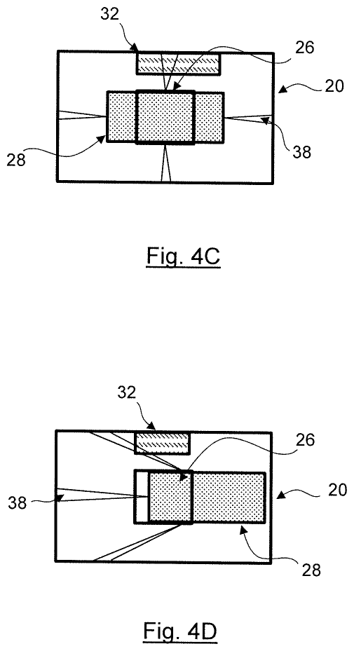

In order to reduce the occurrence of identifying irrelevant or undesirable visual information, the present invention provides that a pointing element 34 may be detected in the captured image(s) (FIG. 4), wherein the processor 16 is adapted to convert the portion of the captured image corresponding to a predefined region 30 of the image relative to the detected pointing element 34 and comprising at least in part the pattern of interest 28.

This is well shown in the embodiment according to FIGS. 4A to 4D. In the example, the pointing element 34 is formed as a pointing device, which may be part of the system 10 as a whole. The provision of the pointing device has the advantage that the patient may use its remaining vision, e.g. remaining peripheral vision and/or remaining central vision, to detect, but not exactly identify, visual information in its presence and focus at the potentially relevant information by placing the pointing device in vicinity of the pattern of interest 28. The generally available hand eye coordination of the patient and potentially available remaining vision facilitate proper placement of the pointing device 34.

Within the captured image, the processor 16 then identifies the pointing device and a corresponding predefined end surface 36. The end surface 36 is exemplarily formed as a truncated portion, which is uniquely distinguishable from an opposing end surface of the pointing device in the longitudinal direction. Other shapes and sizes may be provided as a corresponding end surface 36, which may be identified e.g. using feature recognition algorithms and/or machine learning. Based on the identified predefined end surface 36, the processor 16 converts a predefined region 30 of the captured image into a corresponding input signal for the projector device 14, wherein the predefined region 30 is directly adjacent and centered with respect to the identified predefined end surface 36.

However, as also shown in FIG. 4A, the pointing device and the corresponding predefined region 30 are not at the center of the captured image, i.e. are not aligned with the optical axis of the camera 12. This may result in a reduced focus and quality of the identified pattern of interest 28 perceived by the patient. Particularly when reading a document, this may furthermore be experienced as unnatural by the patient and may limit a fluent and continuous or straight movement of the pointing device along the pattern of interest 28, e.g. when continuing reading.

Therefore, the pattern of interest 28 is cropped during processing into a cropped region 30A, as indicated with the dashed lines in FIG. 4A, and provided with an offset in the signal being output to the projector device 14, wherein said offset corresponds to the offset of the pointing device to the central optical axis of the camera 12. As shown in FIG. 4B, this results in an offset in the laser beam 20 to the top left with regard to the predefined region 30 shown with the dashed lines, as perceived via the modified or, rather, predefined retinal area 22 and indicating to the patient that the camera 12 and head of the patient should be moved in said direction in order to center the pattern of interest 28 and perceive the best possible quality and largest size of the pattern of interest being adjacent to the pointing device.

In order to facilitate proper positioning of the camera 12, the remaining portion of the converted predefined region 30 may be provided as a darkened or lower contrast portion and/or with guidelines 38 originating from the pattern of interest 28 and provided at an angle corresponding with a direction of movement of the head to center the camera.

Alternatively, the offset and/or the guidelines 38 may also be used vice versa, such that the pointing device may be moved in order to provide an initial centering, e.g. when the pointing device is not provided at the appropriate position to identify the pattern of interest 28.

A correction of the position of the camera 12, i.e. by moving the head of the patient, is shown in FIG. 4C, wherein the pattern of interest 28 is perceived at a central position and wherein the guidelines 38 confirm appropriate centering of the camera 12.

If the patient now continues identifying the pattern of interest 28 by moving the pointing element 34 or pointing device into the right direction, the pattern of interest 28 will again be cropped and guidelines 38 will again indicate that a corresponding movement of the camera 12 is required to maintain the pattern of interest 28 at an essentially central position. In such manner, a natural and fluent movement and identification of visual information is facilitated, wherein the guidelines 38 and the offset provide a direct feedback to the patient regarding the positioning of the camera 12. Even if the patient does not readily identify the pattern of interest, e.g. due to a diminished peripheral vision, a diminished hand eye coordination, or a large number of potentially distracting objects 32, the system 10 provides that the identification of the pattern of interest 28 is much easier, such that the occurrence of a patient being lost is significantly reduced.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 System
12 Camera
14 Projector device
16 Processor
18 Camera field of view
20 Light beam
22 Modified or, rather, predefined retinal area
24 Eye pupil
26 Modified or, rather, predefined retinal area image
28 Pattern of interest
30 Predefined region
30A Cropped region
32 Object
34 Pointing element
36 End surface
38 Guide line

1. A system for projecting an optical pattern of interest onto a predefined retinal area of a human eye, comprising
a camera for capturing an image,
a projector device for projecting a light beam into a human eye based on an inputted signal, and
a processor being in communication with the camera and the projector device, the processor being adapted to convert a portion of the captured image into a corresponding signal and to output said signal to the projector device, and
wherein the processor is further adapted to detect a presence of a pointing element in the captured image and to convert the portion of the captured image corresponding to a predefined region of the image relative to the detected pointing element and comprising at least in part the pattern of interest

2. The system according to claim 1, wherein the predefined region is adjacent to a predefined end surface of the pointing element.

3. The system according to claim 2, wherein the processor is adapted to identify the predefined end surface by means of a size, a shape, a texture, a material or tissue type, a level of kurtosis, one or more principal axes, and/or a color of the end surface.

4. The system according to claim 3, wherein the predefined end surface is uniquely identifiable.

5. The system according to claim 4, wherein a size of the predefined region is delimited and/or defined by a size of the light beam and wherein the processor is adapted to align the predefined region with the end surface of the pointing element.

6. The system according to claim 1, wherein the processor is adapted to determine a first offset of the pointing element to a central optical axis of the camera, wherein the processor is further adapted to base the signal on said first offset and to define a second offset of the predefined region or the pattern of interest to the center of the light beam corresponding to said first offset in said signal.

7. The system according to claim 1, wherein the processor is adapted to determine a first offset of the predefined retinal area to a central optical axis of the light beam, based on a received orientation information of the predefined retinal area, wherein the processor is adapted to base the signal on said first offset and to define a second offset of the predefined region or the pattern of interest to the center of the light beam corresponding to said first offset in said signal.

8. The system according to claim 6, wherein the processor is adapted to crop the predefined region or the pattern of interest in accordance with the second offset and the size of the light beam

9. The system according to claim 8, wherein the processor is adapted to provide the signal defining a predefined and/or standardized pattern for a portion of the light beam not corresponding to the predefined region or the pattern of interest.

10. The system according to claim 9, wherein, in comparison with the pattern of interest, the predefined and/or standardized pattern is a pattern with reduced brightness, a blurred pattern, a pattern with reduced resolution, an essentially homogeneous pattern, and/or an essentially continuous pattern.

11. The system according to claim 10, wherein the predefined and/or standardized pattern comprises one or more guidelines originating from the predefined region or the pattern of interest and indicating a direction for centering the camera and/or the predefined retinal area.

12. The system according to claim 1, wherein a portion of the signal corresponding to the predefined region only defines the pattern of interest.

13. The system according to claim 1, preceding wherein the pointing element is a hand-held pointing device.

14. The system according to claim 13, wherein the processor is adapted to be communicatively coupled to the pointing device and to determine the predefined region and/or the pattern of interest in the image based on a received orientation information of the pointing device.

15. The system according to claim 1, further comprising a pointing device as the pointing element.
